(19) United States
(12) Patent Application Publication
(10) Pub. No.: US 2007/0009646 A1
(43) Pub. Date: Jan. 11, 2007

(54) Products comprising corn oil and corn meal obtained from high oil corn

(75) Inventors: James Francis Ulrich, Highwood, IL (US); Neal Torrey Jakel, Lake Zurich, IL (US); Daniel Jeffrey Dyer, Kideer, IL (US); Troy Thomas Lohrmann, Lake Villa, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Renessen LLC, Deerfield, IL (US)

(21) Appl. No.: 11/479,293

(22) Filed: Jun. 30, 2006

Related U.S. Application Data
(62) Division of application No. 09/637,843, filed on Aug. 10, 2000, now abandoned.
Application No. 09/637,843 is a continuation-in-part of application No. 09/249,280, filed on Feb. 11, 1999, now Pat. No. 6,313,328.

(51) Int. Cl. A23K 1/00

(52) U.S. Cl. 426/623; 426/635

(57) ABSTRACT

Corn oil and corn meal obtained from high oil corn are included in useful products. The corn oil is extracted from the high oil corn to form the corn meal. The corn oil generally comprises levels of nutrients not found in commercially available corn oils, since most or all of the corn grain, rather than just the germ, is exposed to the extraction process. The corn grain generally includes the steps of flaking corn grain having a total oil content of at least about 8% and extracting a corn oil from the flaked corn grain. The corn oil is useful for making nutritionally enhanced edible oil or cooking oil, lubricants, biodiesel, fuel, cosmetics and oil-based or oil-containing chemical products. The extracted corn meal is useful for making enhanced animal feed rations, snack food, blended food products, cosmetics, and fermentation broth additive.

The present continuation-in-part application claims the priority of copending U.S. patent application Ser. No. 09/249,280 filed Feb. 11, 1999, the entire disclosure of which is hereby incorporated by reference.

The present invention relates to products that are derived from oil and meal obtained from extracted high oil corn.

Corn, Zea mays L., is grown for many reasons including its use in food and industrial applications. Corn oil and corn meal are two of many useful products derived from corn.

Commercial processing plants utilizing conventional methods for extracting corn oil from conventional corn separate the corn seed into its component parts, e.g., endosperm, germ, tipcap, and pericarp, and then extract corn oil from the corn germ fraction. Corn germ produced by wet or dry milling is processed either by pressing the germ to remove the oil or by flaking the germ and extracting the oil with a solvent. In both processes, because the germ was separated from the remainder of the kernel, many or all of the valuable components of the endosperm fraction are absent from the oil.

A corn-based feed product known as hominy feed is obtained from the dry milling process and is a mixture of corn bran, corn germ, and endosperm, and has a minimum of 4% oil. Several steps including cracking, grinding, sieving, and blending are required to manufacture hominy feed and the resulting particle size of hominy feed is small relative to meal made by the extraction method described herein.

Industry and health advocates are continually in search of more nutritious products derived from corn, since products derived from conventional corn lack some desired nutritional components. Thus, there exists a need for improved products derived from corn oil and corn meal.

### SUMMARY OF THE INVENTION

Finished products containing corn oil and/or corn meal obtained from conventional corn include, for example, cooking oil, animal feed, paper and paper products, numerous food products such as salad dressings, extruded and/or puffed snack foods, products containing corn sweeteners, cereals, chips, puddings, candies, and breads.

One aspect of the invention provides a nutritious animal feed comprising the corn meal remaining after extraction of oil from high oil corn. The animal feed can comprise other nutritious products such as vitamins, minerals, high oil seed-derived meal, meat and bone meal, salt, amino acids, feather meal, and many others used in the art of feed supplementation. The animal feed composition can be tailored for particular uses such as for poultry feed, swine feed, cattle feed, equine feed, aquaculture feed, pet food and can be tailored to animal growth phases. Particular embodiments of the animal feed include growing broiler feed, swine finishing feed, and poultry layer finishing feed. Feed products can be made with the extracted corn meal that will have a higher relative percentage of protein and lower relative percentage of oil than similar products made with conventional corn.

Some embodiments of the invention include those wherein: 1) the corn meal has a fiber content of about 3%, a starch content of about 65%, and a protein content of about 12%, at a moisture content of about 10%; 2) the high oil corn grain has a total oil content of at least about 8% wt.; at least about 14% wt., at least about 12% wt., at least about 10% wt., or from about 8% to about 30% wt.; 3) the corn grain being flaked is whole corn grain or cracked corn grain; 4) the corn grain has been subjected to an oil extraction process such as solvent extraction, hydraulic pressing, or expeller pressing, aqueous and enzyme extraction; 5) the high oil corn grain has a total protein content of at least about 7% wt., at least about 9% wt., at least about 11% wt., or from about 7% to about 20% wt.; 6) the high oil corn grain has a total lysine content of at least about 0.15% wt., at least about 0.5% wt., or from about 0.25% wt. to about 2.0% wt.; and/or 7) the high oil corn grain has a total tryptophan content of at least about 0.03% wt., at least about 0.20% wt., or from about 0.03% wt. to about 2.0% wt..

Another aspect of the invention provides a corn oil-based product comprising corn oil obtained by extraction of at least the endosperm and germ of high oil corn. The corn oil-based product can comprise other components such as vinegar, spices, vitamins, salt, hydrogen to form hydrogenated products, and water. The corn oil used in the products of the invention will generally contain a higher proportion of β-carotene, xanthophylls or tocotrienol than similar products made with corn oil extracted from conventional corn employing conventional methods. The corn oil, used in the products of the invention, is generally produced by exposing the entire corn grain, the cracked corn grain or the flaked corn grain to extraction without separation of the germ from the endosperm. Therefore, the solvent-extractable nutrients present in the endosperm are extracted into the corn oil that has been extracted from the germ and endosperm. Products that can be made with the oil prepared as described herein include, but are not limited to, salad dressings, cooking oils, margarines, spray-coated food or feed products, breads, crackers, snack foods, lubricants, and fuels.

Other embodiments of the invention include those wherein: 1) high oil corn grain is cracked, conditioned, flaked and extracted with a solvent; 2) the high oil corn grain has a total oil content of at least about 8% wt.; at least about 14% wt., at least about 12% wt., at least about 10% wt., or from about 8% to about 30% wt.; 3) the corn oil is extracted by pressing cracked corn; 4) the corn oil is extracted by subjecting flaked corn grain to a solvent-based extraction process; 5) the solvents used to extract miscible or soluble substances from the flaked grain include all forms of commercially available hexane, isopropyl alcohol, ethanol, supercritical carbon dioxide or mixtures thereof; 6) the extracted corn oil is provided as miscella; 7) the corn oil is refined by additional processing; and 8) the corn oil is extracted by subjecting flaked corn grain to hydraulic pressing and/or expeller pressing, aqueous and/or enzyme extraction processes.

A third aspect of the invention provides a method of using extracted corn meal in an animal feed ration comprising the step of: 1) providing an extracted corn meal prepared by at least flaking high oil corn to form flaked corn and extracting the flaked corn to remove a portion of the corn oil therefrom; and 2) including the extracted corn meal in an animal feed ration.

A fourth aspect of the invention provides a method of using an extracted corn oil in a food product comprising the steps of: 1) providing an extracted corn oil obtained by at least flaking high oil corn to form flaked corn and extracting the flaked corn to remove a portion of the corn oil therefrom and form the extracted corn oil; and 2) including the extracted corn oil in a food product.

A fifth aspect of the invention provides a method of using extracted corn oil as a feedstock in an oil refining process. The method comprises the steps of: 1) providing an extracted crude corn oil obtained by at least flaking high oil corn to form flaked corn and extracting the flaked corn to remove a portion of the corn oil therefrom and form the extracted crude corn oil; and 2) including the extracted crude corn oil in a raw material stream of an oil refining process.

A sixth aspect of the invention provides various methods of forming extracted blended meals. A first embodiment of this aspect of the invention provides a method of forming an extracted blended meal comprising an extracted meal obtained from high oil corn and one or more other oilseed meals, the method comprising the step of: 1) combining high oil corn grain and one or more other oilseed grains to form a grain mixture; and 2) subjecting the grain mixture to flaking and an extraction process to remove oil therefrom and form the extracted blended meal. A second embodiment provides a method comprising the steps of: 1) combining a cracked and conditioned high oil corn with a cracked and conditioned other oilseed to form a conditioned mixture; 2) flaking the conditioned mixture to form a flaked mixture; and 3) subjecting the flaked mixture to an extraction process to remove oil therefrom and form the extracted blended meal. A third embodiment provides a method comprising the steps of: 1) combining a cracked, conditioned and flaked high oil corn with a cracked, conditioned and flaked other oilseed to form a flaked mixture; and 2) subjecting the flaked mixture to an extraction process to remove oil therefrom and form the extracted blended meal. A fourth embodiment provides a method comprising the step of combining an extracted corn meal with one or more extracted other oilseed meals to form a blended meal, wherein the extracted corn meal has been obtained by at least flaking and extracting high oil corn to form the extracted corn meal. A fifth embodiment provides a blended extracted meal product prepared according to any one of the above-described methods.

A seventh aspect of the invention provides a method of using extracted corn oil as an ingredient in cosmetic applications. The method comprises the steps of: 1) providing an extracted crude corn oil obtained by at least flaking high oil corn to form flaked corn and extracting the flaked corn to remove a portion of the corn oil therefrom and form the extracted crude corn oil; and 2) including the extracted crude corn oil a cosmetic product. These types of cosmetics include but are not limited to lip stick and eye liner.

Another aspect of the invention provides the use of a corn meal in an animal feed or human food, wherein the corn meal is obtained after extraction of corn oil from whole kernels of high oil corn.

Yet another aspect of the invention provides the use of a corn oil in an animal feed or human food, wherein the corn oil is obtained by extraction from whole kernels of high oil corn.

Other aspects of the invention provide corn oil-containing and/or corn meal-containing products made by the processes described herein.

Unless otherwise defined, all technical and scientific terms and abbreviations used herein have the same meaning as commonly understood by one of ordinary skill in the art to which this invention pertains. Although methods and materials similar or equivalent to those described herein can be used in the practice of the present invention, suitable methods and materials are described below without intending that any such methods and materials limit the invention described herein. All patents publications and official analytical methods referred to herein are incorporated by reference in their entirety. Additional features and advantages of the invention will be apparent from the following description of illustrative embodiments of the invention and from the claims.

### DETAILED DESCRIPTION OF THE INVENTION

It has been discovered that corn oil can be rapidly and efficiently extracted on a commercial-scale from corn grain having increased oil content by optionally cracking and then conditioning, and flaking the corn grain and extracting a corn oil. Useful corn grain for the novel flaking oil processing method has a total oil content greater than about 8%. Increases in the oil content of corn grain may increase flaking efficiency during processing. Suitable flaking equipment and methods include conventional flaking equipment and methods used for flaking soybean and other similar oilseed types. Suitable extracting equipment and methods may include conventional methods used-for extracting oil from soybean flakes and other similar oilseed types.

High oil corn seed or “grain” harvested from any of a number of different types of corn plants is useful in the invention. Such corn plants may be hybrids, inbreds, transgenic plants, genetically modified plants or a specific population of plants. Enhanced extracted meals can be made by subjecting enhanced high oil corn to the extraction process described herein. Useful corn grain types include, for example, flint corn, popcorn, flour corn, dent corn, white corn, and sweet corn. The high oil corn grain can be in any form including whole corn, cracked corn, or other processed corn or parts thereof that are amenable to flaking but different from the standard methods of germ separation employed in dry and wet milling for subsequent recovery of oil from the germ.

As used herein, the terms “whole kernel” or “whole corn” mean a kernel that has not been separated into its constituent parts, e.g. the hull, endospemn, tipcap, pericarp and germ have not been purposefully separated from each other. The whole corn may or may not have been ground, crushed, cracked, flaked, or abraded. Purposeful separation of one corn constituent from another does not include random separation which may occur during storage, handling, transport, crushing, flaking, cracking, grinding or abrading. A purposeful separation of the constituent part is one wherein at least 50% of one constituent, e.g., germ, has been separated from the remaining constituents.

As used herein, the term “high oil corn” refers to corn grain comprising at least about 8% wt. oil. A high oil corn has an elevated level of oil as compared to conventional yellow dent corn, which has an oil content of about 3% to about 5% wt. Additionally, the total oil content of corn grain suitable for the invention can be, for example, grain having an oil content at least about 9%, at least about 11%, at least about 12%, at least about 15%, at least about 18%, at least about 20%, from about 8% to about 20% oil, from about 10% to about 30% oil, or from about 14% to about 30%, and values within those ranges. Although the oil content can be determined at any moisture content, it is acceptable to normalize the oil content to a moisture content of about 15.5%.

High oil corn useful in making the oil and meal described herein are available from Cargill, Incorporated (Minneapolis, Minn.) or Pfister Hybrid Corn Co. (El Paso, Ill.). Other suitable high oil corn includes the corn populations known as Illinois High Oil (IHO) and Alexander High Oil (Alexo), samples of which are available from the University of Illinois Maize Cooperative Stock Center (Champaigne-Urbana, Ill.) Corn grain having an elevated total oil content is identified by any of a number of methods known to those of ordinary skill in the art. The oil content of grain, including the fat content of a meal extracted from the grain, can be determined using American Oil and Chemical Society Official Method, 5^{th} edition, March 1998, (“AOCS method”) Ba 3-38. AOCS method BA 3-38 quantifies substances that are extracted by petroleum ether under conditions of the test. The oil content or concentration is the weight percentage of the oil with respect to the total weight of the seed sample. Oil content may be normalized and reported at any desired moisture basis.

Other suitable methods for identifying high oil corn grain are described herein. According to one method, corn ears are selected using a near infrared (NIR) oil detector to select corn ears having corn kernels with elevated oil levels. Likewise, an NIR detector can also be used to select individual corn kernels having elevated levels of corn oil. However, selecting individual ears and/or kernels having elevated oil content may not be cost effective to identify high oil kernels suitable for processing using methods described herein. Generally, corn seed producing corn plants that yield grain having elevated total oil concentrations is planted and harvested using known farming methods. Methods for developing corn inbreds, hybrids, transgenic species and populations that generate corn plants producing grain having elevated oil concentrations are known and described in (Lambert, 1994. In: Specialty Corns. A. R. Hallauer, ed., High Oil Corn Hybrids. pp. 123-145. CRC Press. Boca Raton, Fla., USA).

One of the suitable high oil corns used as a raw material for preparing the corn oil and corn meal used in the invention has a nutrient profile as shown in Table 1. Amounts are expressed on an “as is” or “as fed” moisture level. Protein, oil, and starch levels can vary in a number of possible combinations in the high oil corn used as a raw material for meal and oil used in the invention. Acceptable amounts of moisture, oil, protein, starch, lysine, and tryptophan are illustrated in Table 1. However, additional combinations, such as 12% protein and 12 % oil, not shown as indicated amounts in the table are within the scope and range of corn grain to be used to produce oil and meal used in the invention.

TABLE 1
Exemplary amounts and general profiles of high oil corns used
for preparing the corn oil and corn meal used in the invention.

| | Amount 1 | Amount 2 | Amount 3 | General |
|---|---|---|---|---|
| Component | (%) | (%) | (%) | Amount (%) |
| Moisture | 14 | 14 | 14 | 5-45 |
| Oil | 8 | 12 | 20 | 8-30 |
| Protein | 9 | 9 | 17 | 5-20 |
| Starch | 61 | 54 | 41 | 35-80 |
| Lysine | 0.35 | 0.50 | 1.0 | 0.15-2.0 |
| Tryptophan | 0.088 | 0.11 | 0.15 | 0.03-2.0 |

Another suitable high oil corn used as a raw material for preparing the corn oil and corn meal used in the invention has a nutrient profile as shown in Table 2. Amounts are expressed on an “as is” or “as fed” moisture level. The amounts shown in Table 2 are exemplary for a corn grain having 12% oil and 9% protein.

TABLE 2

| Component | Amount (%) | General Amount (%) |
|---|---|---|
| Moisture | 14 | 5-45 |
| Oil | 12 | 8-30 |
| Protein | 9 | 5-20 |
| Starch | 65 | 35-80 |
| Fiber | 3 | 1-5 |
| Ash | 1.18 | 0.59-4.72 |
| Lysine | 0.33 | 0.2-2.0 |
| Tryptophan | 0.09 | 0.03-2.0 |
| Methionine | 0.25 | 0.13-1.00 |
| Total Sulfur Amino Acids | 0.46 | 0.23-1.84 |
| Valine | 0.45 | 0.23-1.80 |
| Isoleucine | 0.34 | 0.17-1.36 |
| Arginine | 0.45 | 0.23-1.80 |
| Threonine | 0.34 | 0.17-1.36 |
| Leucine | 1.03 | 0.52-4.12 |
| Histidine | 0.27 | 0.14-1.08 |
| Phenylalanine | 0.44 | 0.22-1.76 |
| Alanine | 0.70 | 0.35-2.80 |
| Aspartic | 0.74 | 0.37-2.96 |
| Cystine | 0.22 | 0.11-0.88 |
| Glutamic | 1.9 | 0.95-7.6 |
| Glycine | 0.46 | 0.23-1.84 |
| Proline | 0.86 | 0.43-3.44 |
| Tyrosine | 0.06 | 0.03-0.54 |
| Serine | 0.46 | 0.23-1.84 |

Table 3 shows amino acid levels of two high oil corn grain samples and normal yellow corn grain. The oil and protein levels of high oil corn sample 1 (HOC 1) were 13.3% and 10.7% respectively, expressed on a dry matter basis. The oil and protein levels of high oil corn sample 2 (HOC 2) were 13.0% and 11.2% respectively, expressed on a dry matter basis. For comparison, normal yellow corn grain has about 4.2% oil and about 9.2% protein on a dry matter basis.

TABLE 3
Amino acid profiles of two high oil corn grain samples, HOC 1 and
HOC 2, and normal yellow corn grain. Levels are expressed
based on a corn grain moisture content of about 10%.

| Amino Acid | HOC 1 (%) | HOC 2 (%) | Yellow Corn (%) |
|---|---|---|---|
| Aspartic Acid | 0.71 | 0.68 | 0.48 |
| Threonine | 0.33 | 0.30 | 0.19 |
| Serine | 0.37 | 0.27 | 0.19 |
| Glutamic Acid | 1.84 | 1.79 | 1.16 |
| Proline | 0.83 | 0.78 | 0.52 |
| Glycine | 0.40 | 0.42 | 0.24 |
| Alanine | 0.77 | 0.74 | 0.47 |
| Valine | 0.51 | 0.52 | 0.33 |
| Cystine | 0.21 | 0.23 | 0.16 |
| Methionine | 0.46 | 0.47 | 0.39 |
| Isoleucine | 0.30 | 0.30 | 0.20 |
| Leucine | 1.19 | 1.08 | 0.74 |
| Tyrosine | 0.11 | 0.11 | 0.06 |
| Phenylalanine | 0.52 | 0.48 | 0.32 |
| Tryptophan | 0.06 | 0.07 | 0.05 |
| Lysine | 0.34 | 0.38 | 0.21 |
| Histidine | 0.29 | 0.29 | 0.18 |
| Arginine | 0.45 | 0.48 | 0.28 |

The high oil corn is generally subjected to an extraction process as described herein in order to provide the enhanced corn oil and corn meal to be included in the finished products of the invention. As used herein, the term “finished product” or “product” refers to a product made by including the corn oil and/or corn meal of the invention in combination with a variety of other ingredients. The specific ingredients included in a product will be determined according to the ultimate use of the product. Exemplary products include animal feed, raw material for chemical modification, biodegradable plastic, blended food product, edible oil, cooking oil, lubricant, biodiesel, snack food, cosmetics, and fermentation process raw material. Products incorporating the meal described herein also include complete or partially complete swine, poultry, and cattle feeds, pet foods, and human food products such as extruded snack foods, breads and as a food binding agent, aquaculture feeds, fermentable mixtures, food supplement, sport drink, nutritional food bar, multi-vitamin supplement, diet drink and cereal foods.

For example, starting with a single corn type (12% oil and 9% protein), more than one meal type can be made to meet certain nutritional requirements. The significance of this flexibility relates to the nutrient density within feed products and to dietary requirements of animals. One significant advantage of the use of this type of high oil corn and extraction process is that an extracted corn meal can be made to have a specific oil level depending on the extent of oil extraction. Once the oil is removed from the flakes, the remaining meal has a nutrient density for protein, amino acids, and other nutrients not removed by the process, greater or different than normal corn grain, and greater than that of the starting corn, e.g., 12% oil, 9% protein.

According to one extraction process used in preparing the corn oil and corn meal as described herein, whole high oil corn is optionally cracked and then conditioned and flaked. After flaking, the flaked corn is extracted as described herein.

The high oil corn is cracked by passing the whole grain corn between two rollers with corrugated teeth spinning toward each other spaced by a defined gap, and/or passing through a grind mill where a rotating toothed disk spins at an adjustable distance from a stationary disk, and/or the use of a hammermill where two rotating metal “hammer” like devices spinning next to one another. Methods for cracking corn or high oil seeds are described in (Watson, S. A. & P. E. Ramstad, ed. 1987 In: Corn: Chemistry and Technology, Chapter 11, American Association of Cereal Chemist, Inc., St. Paul, Minn. USA), the disclosure of which is hereby incorporated by reference in its entirety. A cracked corn is a corn that has undergone the above-described cracking process.

Whether or not the corn is cracked, it may be conditioned using methods known to those of ordinary skill in the art or methods described herein. As used herein, the term “conditioning” refers to a process by which the corn kernel is softened or plasticized to render it more pliable and amenable to the flaking and extraction processes. Conditioning may consist of the addition of steam (saturated and/or non-saturated steam) and/or water to the cracked high oil corn. This is done by the use of a rotary conditioner. Both the temperature and moisture levels are elevated. Temperature ranges between about 140 degrees F. and about 210 degrees F. and the moisture is increased by about 1% to about 15%.

The high oil corn grain is then flaked to any useful size. As used herein, the term “flaking” refers to a process by which corn grain is passed one or more times through flaking rollers to produce flakes. The flaked corn may have a final flake thickness of about 5/1000-50/1000 of an inch or about 0.12 mm-1.0 mm or about 0.01 inches (0.25 mm), although other thickness may also be used. Useful flake thickness may depend on external limiting parameters such as the oil content of the corn, the moisture content, the corn type, e.g., dent or flint, and the oil extractor type. Suitable methods for flaking high oil corn are detailed herein and in D. R. Erickson, Practical Handbook of Soybean Processing Utilization (1995, AOCS Press), the entire disclosure of which is hereby incorporated by reference. Suitable flaking methods also include those known to those of ordinary skill in the art of oilseed processing.

After the corn is cracked and/or conditioned and flaked, the flaked corn is subjected to an extraction process in order to extract oil to form an extracted corn meal (ECM). Corn oil is extracted from flaked grain by one or more extraction steps using any extraction method. Generally, substantially or about all of the oil is extracted in a single extraction process. Useful extraction methods include solvent extraction, hydraulic pressing, expeller pressing, aqueous and/or enzyme extraction. Useful solvents for solvent extraction include, for example, all forms of commercially available hexane, isopropyl alcohol, ethanol, supercritical carbon dioxide, combinations thereof and other similar solvents. For example, corn oil can be extracted from flaked grain using a hexane-based solvent extractor. Solvent extractors can include both percolation and immersion type extractors.

Materials removed from solvent-based extractors include wet flakes and miscella. A miscella is a mixture of extracted oil and solvent. The wet flakes are the materials that remain after some or all of the solvent-soluble material has been extracted. Wet flakes also contain a quantity of solvent. Solvent is reclaimed from both the miscella and wet flakes using methods such as rising film evaporation, or drying, and raising the temperature using equipment such as flash tanks and/or de-solventiser/toasters. For example, heat is applied to the wet flakes or miscella under atmospheric pressure, under elevated pressure, or under vacuum to evaporate the solvent. The evaporated solvent is then condensed in a separate recovery system.

Desolventized miscella are referred to as crude oil, which can be stored and/or undergo further processing. Crude oil can be refined to produce a final oil product. Methods for refining crude oil to obtain a final oil are known to those of ordinary skill in the art. Hui (1996) provides a thorough review of oils and oilseeds (Hui, Y. H., ed. 1996. In: Bailey's Industrial Oil and Fat Products. Fifth Ed., Vol. 2: Edible Oil and Fat Products: Oils and Oilseeds. John Wiley and Sons, Inc., New York). Chapter three of Hui (1996, pp. 125-158; the disclosure of which is hereby incorporated by reference) specifically describes corn oil composition and processing methods. Crude oil isolated using the flaking methods described herein is of a high quality but can be further purified as needed using conventional oil refining methods.

Corn endosperm includes some valuable components such as carotenoids, lutein, and zeaxanthin. Carotenoids in grains are classified into two general groups, the carotenes and the xanthophylls. The carotenes are important because they are vitamin A precursors. Blessin et al., 1963 (Cereal Chem. 1963. 40:582-586) found that over 90% of the carotenoids, of which beta-carotene is predominant, are located in the endosperm of yellow dent corn and less than 5% are located in the germ. Vitamin A is derived primarily from beta-carotene.

Another group of valuable components found in the endosperrn includes the tocotrienols. Grams et al., 1970, discovered that in corn, tocotrienols were found only in the endospern, whereas the germ contained most of the tocopherols. Tocotrienols can be extracted from plant material using various solvents. Processes for recovering tocotrienols from plant material are described by Lane et al. in U.S. Pat. No. 5,908,940, the entire disclosure of which is incorporated by reference.

One embodiment of the invention provides an extracted corn oil having greater amounts of lutein, zeaxanthin and beta-carotene than does commercially available crude oil obtained from commodity normal yellow #2 dent corn. Conventional crude oil can be obtained from suppliers such as Cargill, Incorporated (Minneapolis, Minn.). For example, a corn oil prepared according to Example 1 comprised the following ingredients in the amounts indicated as compared to commercially available crude oil.

| | Lutein | Zeaxanthin | Beta-Carotene |
|---|---|---|---|
| Sample | (mg/g) | (mg/g) | (IU/100 g) |
| Commercial Crude | 0.005 | 0.005 | 15.5 |
| Corn Oil | | | |
| Oil Sample 1 | 0.04 | 0.012 | 72.3 |
| Oil Sample 2 | 0.330 | 0.112 | 302 |

Accordingly, the process described herein provides a nutritionally enhanced corn oil enriched with lutein, zeaxanthin, and/or beta-carotene and optionally one or more other nutritional components.

Oil-based products made with corn oil obtained by the extraction method described herein can contain higher levels of important nutrients than similar products made with corn oil produced by conventional methods. The corn oil obtained by the extraction methods described herein will include the corn oil from the germ and endosperm, and one or more other components extracted from the rest of the kernel. The one or more other components can be oil from the endosperm, tocotrienols, tocopherols, carotenoids, carotenes, xanthophylls, and sterols.

Tocopherols (vitamin E) and vitamin A are antioxidants and are fat-soluble vitamins. When included in the diet, both have demonstrated health benefits. Blending of oil of the present invention with other oils or substances to achieve an appropriate level of beta-carotene, vitamin E, and tocotrienols is deemed within the scope of the present invention. In some embodiments, extracted corn oil prepared as described herein comprises about 0.1% wt. to about 0.5% wt. of tocopherol.

Oil with approximately a 200% to 300% increase in tocotrienol content over conventionally-produced crude corn oil is described. Using the method of cracking and/or conditioning and/or flaking and extraction of high oil corn the corn oil was extracted and was then analyzed for tocotrienol content. The actual minimum and maximum values for tocotrienol content will depend upon the particular high oil corn used.

The oxidative stability index (OSI), which is measured in hours, is a measure of an oil's relative stability toward oxidation. Generally, the greater the OSI, the less susceptible the oil is toward oxidation and the longer it takes to oxidize the oil under test or use conditions. In addition, the greater the content of unsaturated fatty acids present in the oil, the lower the OS. Exemplary oils prepared according to the extraction method described herein generally possess OSI values ranging from about 10-22 hours.

Extraction of carotenes and xanthophylls and other pigments is described in detail by Blessin (Cereal Chem. 1962. 39:236-242; the entire disclosure of which is incorporated by reference). Combinations of solvents, primarily ethanol and hexane, can be used to extract carotenes and xanthophylls from corn. Ethanol, hexane, other solvents combinations and ratios thereof may be used to produce oil of the present invention on a commercial scale.

Exemplary embodiments of the crude oil obtained according to the extraction method described herein generally possess the following partial composition profile.

| | Exemplary | |
|---|---|---|
| | Extracted High | Extracted High |
| Component | Oil Corn | Oil Corn (Range) |
| FFA (%) | 1.45 | 0.7-3.00 |
| C16:0 | 11.4 | 10-14 |
| C18:0 | 2.1 | 1.5-3.5 |
| C18:1, cis | 33 | 26-50 |
| C18:1, trans | | |
| C18:2, cis | 50 | 42-60 |
| C18:2, trans | | |
| C18:3 | 0.8 | 0.6-1.6 |
| Total trans | | |
| Phosphorous (ppm) | 190 | 100-400 |
| Total Tocopherols | 0.13 | 0.1-.50 |
| (ppm) | | |

Fatty acids generally found in the corn oil generally include palmitic, stearic, oleic, linoleic and linolenic acids.

The crude oil prepared according to the methods described herein can be subsequently partially or completely hydrogenated. Suitable methods for partially or completely hydrogenating oil are described in D. R. Erickson, Practicle Handbook of Soybean Processing utilization (1995, AOCS Press), the entire disclosure of which is hereby incorporated by reference.

When making oil-based products according to the invention, those products can include conventional corn oil, soy oil, canola oil, olive oil, palm oil, sunflower oil, safflower oil, antioxidant, flavoring, hydrogenated oil, partially hydrogenated oil and/or animal fat. By mixing the corn oil herein with one or more other oils, blended oil products are made. The corn oil-based products can also include materials such as food additives, salt, fat, food colors, β-carotene, annatto extract, curcumin or tumeric, β-apo-8′-carotenal and methyl and ethyl esters thereof, natural or synthetic flavors, antioxidants, propyl gallate, butylated hydroxytoluene, butylated hydroxyanisole, natural or synthetic tocopherols, ascorbyl palmitate, ascorbyl stearate, dilauryl thiodiproprionate, antioxidant synergists, citric acid, sodium citrate, isopropyl citrate, phosphoric acid, monoglyceride citrate, anti-foaming agent, dimethyl polysiloxane, crystallization inhibitor, oxystearin, amino acids, vitamin, minerals, carbohydrates, sugars, herbs, spices, acidity regulators, firming agents, enzyme preparations, flour treatment agents, viscosity control agents, enzymes, lipids, and/or vegetable or animal protein. Additionally, these edible products can be enhanced or enriched with protein supplements containing utilizable protein. An exemplary food product such as a breakfast cereal could include ingredients such as meal of the invention, wheat and oat flour, sugar, salt, corn syrup, milled corn, dried fruit, vitamin C, B vitamins, folic acid, baking soda, and flavorings.

Other exemplary oil-based products that can comprise the oil prepared herein include food oil, cooking oil, edible oil and blended oil.

Equipment used for the extraction of oil from oilseeds, such as soybean and canola, can be used to prepare the corn oil and extracted corn meal described herein. Useful commercial-scale oilseed flakers can be obtained from French Oil Mill Machinery Company, Piqua, Ohio USA 45456-0920; Roskamp Champion, Waterloo, Iowa; Buhler, based in Switzerland and having offices in Plymouth, Minn. USA; Bauermeister, Inc., Germany; and Consolidated Process Machinery Roskamp Company, on the world wide web at http://www.cpmroskamp.com, and Crown Iron Works, Minneapolis, Minn.

Commercial-scale methods and equipment are sufficient for extracting corn oil from at least about 1 ton of corn per day. In some embodiments, the capacity of commercial-scale operations ranges from about 100 tons of corn per day to about 3000 tons of corn per day, or the capacity ranges from about 700 tons of corn per day to about 1700 tons of corn per day. Commercial-scale operations that process greater than about 3000 tons of corn per day are also sufficient.

Corn oil or corn meal quality is determined by evaluating one or more quality parameters such as the oil yield, phosphorous content, free fatty acid percentage, the neutral starch percentage, protein content, and moisture content. Any method can be used to calculate one or more of the quality parameters for evaluating the oil or meal quality.

The phosphorous concentration of crude oil can be determined using AOCS method Ca 12-55. AOCS method Ca 12-55 identifies the phosphorous or the equivalent phosphatide zinc oxide, followed by the spectrophotometric measurement of phosphorous as a blue phosphomolybdic acid complex. AOCS method Ca 12-55 is applicable to crude, degummed and refined vegetable oils. The phosphorous concentration is converted to phospholipid concentration, i.e., gum concentration, by multiplying the phosphorous concentration by 30. ln some embodiments, corn oil produced according to the invention includes about 100-400 ppm of phosphorous.

The free fatty acid percentage of oil can be determined using AOCS method Ca 5a40. AOCS method Ca 5a-40 identifies the free fatty acids existing in the oils sample. AOCS method Ca 5a-40 is applicable to all crude and refined vegetable oils, marine oils and animal fats. The neutral oil loss during processing is determined by adding the gum percentage and the free fatty acid percentage together. The amount of free fatty acid obtained in the extracted corn oil will depend upon the amount of fatty acids found within the high oil corn from which the oil was extracted. In some embodiments, the free fatty acid content of the extracted oil ranges from about 0.70%-3.00% wt.

Oil color can be determined using AOCS method Cc 13b-45. AOCS method Cc 13b45 identifies the color of an oil sample by comparing the oil sample with known color characteristics. AOCS method Cc 13b-45 is applicable to fats and oils provided no turbidity is present in the sample. Color values are evaluated qualitatively by visual inspection of the oil. Generally, visual inspection results in an oil being classified as a light oil or a dark oil compared to a known oil color. Color values are quantitated by determining a red color value and a yellow color value using the AOCS method Cc 13b-45. Typically, crude corn oil isolated using conventional dry milling methods has a red color value ranging from about 7 to about 10 and a yellow color value ranging from about 60 to about 70. Corn oils isolated using flaking methods described herein have oil colors that qualitatively are considered light and is generally lighter than crude corn oil color made using wet or dry milling techniques. The yellow color values may range from about 60 to about 70 and red color values may range from about 7 to about 10, as determined by American Oil and Chemical Society method Cc 13b-93.

The extracted corn oil can be used as a raw material for chemical modification, a component of biodegradable plastic, a component of a blended food product, a component of an edible oil or cooking oil, lubricant or a component thereof, biodiesel or a component thereof, a component of a snack food, a fermentation process raw material, and a component of cosmetics. Since the oil obtained by the extraction process herein has one or more components obtained from non-germ parts of the corn kernel, the oil is enhanced. In some embodiments, the oil will have an oleic range from about 20% to 80%, or 25% to 50%, where normal corn would have about 25% to 40% oleic acid in the oil. When making blended oils with the extracted oil, the blending can be done before, during or after the extraction process.

Meal produced from the flaking and oil extraction process described herein is used to produce unique feed products. The corn meal used herein has been obtained after extraction of oil from whole kernels of high oil corn, wherein the kernel has not been separated into its constituent part, although the kernel may or may not have been ground, flaked, cracked, chipped, or abraded. The process of removing the oil from corn via extraction serves to concentrate the remaining nutrients such as protein and essential amino acids. Feed products containing predominantly corn meal produced by extraction will require less supplementation with protein from other sources such as soybeans than will feed products containing predominantly normal corn grain. The meal, by virtue of the composition arising from the processing method, offers feed manufacturers flexibility to produce feeds that could otherwise not be made. Animal feed rations having unique physical properties such as bulk density, texture, pelletability, and moisture holding capacity and/or unique nutritional properties are created by including the extracted corn meal of the present invention as a component of said rations. The extracted corn meal isolated using flaking and extraction methods as described herein can by itself, i.e., as is, be a low fat corn meal. Alternatively, it can be used in combination with other meals or nutritional components to make feed rations and food products. The extracted corn meal can also be combined with meals made from crops such as soybeans, canola, sunflower, oilseed rape, cotton, and other crops. The extracted corn meal can also be made from genetically modified corn and/or combined with meals made from transgenic oilseed grains to form an enhanced meal or enhanced product.

The extracted corn meal can be provided as a loose product or a pelleted product, optionally in combination with other components. For example, a pelleted product could include the extracted corn meal (by itself or in combination with other components) that has been pelleted and subsequently coated with zein protein. The corn meal can be included in blended meal products which can be provided in loose or pelleted form.

The feed rations prepared with the extracted corn meal will generally meet the dietary and quality standards set forth in the CODEX ALIMENTARIUS or the National Research Council. The meal will generally comprise the following components in the approximate amounts indicated in the table below.

| | Sample A | Sample B | Sample C |
|---|---|---|---|
| Component | Amount (%) | Amount (%) | Amount (%) |
| Moisture | 5-45 | 5-25 | 5-45 |
| Starch | 40-70 | 40-80 | 40-70 |
| Protein | 8-20 | 7-20 | 8-20 |
| Fat (Oil) | 0.75-6 | 0.75-6.0 | 0.75-12 |
| Crude Fiber | 2-4 | 2-4 | |
| Ash | 1.5-3 | 0.5-2.0 | |
| Fructose | 0.15-0.3 | | |
| Glucose | 0.2-0.5 | | |
| Sucrose | 1.5-2.5 | | |
| Lysine | | | 0.2-2.0 |
| Tryptophan | | | 0.03-2.0 |

The meals above may also further comprise unspecified amounts of the components for which no amounts have been indicated.

When compared to meals made from conventional corn, the extracted corn meal described herein provides a greater amount of some key nutritional components (nutrients) such as vitamins, folic acid, pantothenic acid, lysine, tryptophan, and/or niacin. For example, meal Samples 1 and 2 of extracted corn meal which are prepared according to Example 1 include the following nutritional components in the amounts indicated. Amounts for the same components, to the extent they are found in yellow corn that has not been processed as described herein, are included for comparison.

| Component | Yellow Corn | Meal Sample 1 | Meal Sample 2 |
|---|---|---|---|
| Vitamin B6 | 0.400 | 0.820 | 0.660 |
| (mg/100 g) | | | |
| Vitamin B12 | 0.500 | 0.500 | 0.500 |
| (mg/100 g) | | | |
| Folic Acid | — | 25.0 | 25.0 |
| (μg/100 g) | | | |
| Pantothenic Acid | — | 0.660 | 0.890 |
| (mg/100 g) | | | |
| Niacin | 2.05 | 2.30 | 1.15 |
| (mg/100 g) | | | |

The extracted corn meal prepared as described herein advantageously can be made to contain specific levels of oil and, in particular, specific ratios of oil to protein, of oil to carbohydrate or of oil to protein to carbohydrate. For example, normal corn with 8% protein and 4% oil has a protein:oil ratio of 2.0, and high oil corn with 9% protein and 12% oil has a protein:oil ratio of 0.75. Meal produced by extraction to have 10.5% protein and 1.5% oil has a protein:oil ratio of 7.0. This higher ratio makes this meal type and products made from it desirable for certain applications, one example being a swine finishing ration.

Varying levels of nutrients are required by different animals depending on species, age, and breed. Feed rations comprising different levels of nutrients are made by subjecting the high oil corn to different degrees of extraction, i.e., more oil is removed from the corn by subjecting it to extraction to a greater degree. Therefore, feed rations comprising the extracted corn meal of the invention can be made to include different amounts of fat, protein, and carbohydrates by controlling the extent to which the high oil corn is extracted. The following table details the amounts in which the indicated ingredients are present in animal feed rations comprising the extracted corn meal, the specific inclusion range being indicative of exemplary rations in which extracted corn meal is a main ingredient, and the general inclusion range being indicative of rations in which one or more other ingredients, for example, carbohydrate-based energy sources such as sorghum, wheat, and/or other cereal grains or their by-products, or other non-cereal grain ingredients, may be included.

| | General Inclusion | Exemplary |
|---|---|---|
| Ingredient | Range | Inclusion Range |
| Corn meal described herein | 2-95% | 50-90% |
| Oilseed Meal¹ | 3-35% | 10-30% |
| Meat and Bone Meal | 0-12% | 0-7% |
| Feather Meal | 0-6% | 0-4% |
| Fat | 0-10% | 1-6% |
| Salt | 0.1-0.5% | 0.1-0.5% |
| Lysine | 0-0.4% | 0-0.4% |
| Methionine | 0-0.3% | 0-0.3% |
| Nutrient Premix | 0.01-1.0% | 0.01-1.0% |

¹Oilseed meal can consist of, but is not limited to, soy, sunflower, canola, cottonseed, and other plant-based meals, which themselves may or may not have been subjected to an oil extraction process.

Meat and bone meal is obtained from suppliers such as Darling International, Inc. (Irving, Tex.). Oilseed meal is obtained from suppliers such as Cargill Oilseeds (Cedar Rapids, Iowa). Feather meal is obtained from suppliers such as Agri Trading Corp., (Hetchinson, Minn.). Amino acids are obtained from suppliers such as DuCoa, (Highland, Ill.).

Feed rations are made by mixing various materials such as grains, seed meals, vitamins, and/or purified amino acids together to form a composite material that meets dietary requirements for protein, energy, fat, vitamins, minerals, and other nutrients. The mixing process can include grinding and blending the components to produce a relatively homogeneous mixture of nutrients. Physical properties of the feed raw materials and of the compounded feed affect the nutritional quality, storability, and overall value of the products. Suitable processes for manufacturing feed rations are disclosed in Feed Manufacturing Technology IV, 1994, which is published by the American Feed Industry Association.

The extracted corn meal may be somewhat analogous to steam-flaked corn in terms of digestibility of the starch fraction, but may have better digestibility in ruminants by virtue of the processing conditions. As discussed herein, specific oil levels can be achieved in the extracted meal by altering processing conditions. The protein, amino acid, and oil levels of the present extracted meal cannot be achieved in steam-flaked normal corn, and steam-flaked high oil corn may have too much oil, which could adversely affect ruminant animal health.

Many types of animal feed rations can be developed using extracted corn meal of the present type, and for illustration purposes, the following diet types will be described herein:
1. Meal made from corn grain wherein the said corn grain has an oil content of 12% and a protein content of 9%, and meal resulting from this corn has an oil content of 1.5% for use in a hog finishing diet.
2. Meal made from corn grain wherein the said corn grain has an oil content of 12% and a protein content of 9%, and meal resulting from this corn has an oil content of 4.0% for use in a poultry broiler diet.

Blended products comprising the extracted corn meal and one or more other oilseed meals are made by one or more of the following ways: 1) combining the high oil corn and the other oilseed prior to cracking and/or flaking and subjecting the entire seed mixture to the flaking and extraction process described herein to form a blended meal; 2) combining the high oil corn and the other oilseed after cracking and conditioning, but prior to flaking and subjecting the entire seed mixture to an extraction process as described herein to form a blended meal; 3) combining the high oil corn and the other oilseed after flaking and subjecting the entire seed mixture to the extraction process described herein to form a blended meal; 4) combining the extracted corn meal with extracted or non-extracted other oilseed meal to form a blended meal; or 5) combinations thereof to form a blended meal. At any time during the just described processes, additional components can be added to the blended meals to form a blended product.

The extracted corn meal can also be used in foodstuffs such as snack food, blended food products, breads, fermentation feedstock, breakfast cereals, thickened food products such canned fruit fillings, puffed or extruded foods, and porridge.

When used in edible products for humans or animals, the extracted corn meal can be combined with other components such as other meal, other oilseed meal, grain, other corn, sorghum, wheat, wheat milled byproducts, barley, tapioca, corn gluten meal, corn gluten feed, bakery byproduct, full fat rice bran, and rice hull.

The extracted corn meal can also be used as a raw material for production of corn protein isolates, for fermentation, for further chemical processing, in addition enzymes, such as amylases and proteases, can be added to the meal to help facilitate the breakdown of starch and proteins.

The extracted corn meal is optionally subjected to conventional methods of separating the starch and protein components. Such methods include, for example, dry milling, wet milling, high pressure pumping or cryogenic processes. These and other suitable processes are disclosed in Watson, S. A. & P. E. Ramstad, ed. 1987 In: Corn: Chemistry and Technology, Chapter 11 and 12, American Association of Cereal Chemist, Inc., St. Paul, Minn. USA), the disclosure of which is hereby incorporated by reference. Due to the prior removal of oil from the corn meal, the starch and protein components of the extracted corn meal are separated from other components more easily than they would be if the corn oil were not extracted.

Several important quality parameters for the extracted meal include the fat, starch, protein, and moisture content. Methods for evaluating quality parameters of oilseed meals are disclosed in the AOCS methods, the relevant disclosure of which is hereby incorporated by reference. These methods can also be applied to the extracted corn meal prepared as described herein.

The moisture content of the grain can affect the flaking process. It may be necessary for the moisture of the corn grain to be increased by about 1% to about 15% before flaking the seed. Optimizing the grain moisture content to facilitate efficient processing is within the knowledge of those of ordinary skill in the art.

Corn meals derived using different methods or isolated at different times are compared by normalizing the meals to a common moisture content. The moisture content of an oilseed protein concentrate, such as a corn meal or whole corn, is determined using AOCS method Ba 2b-82. The crude fiber content of corn meal is determined using AOCS method Ba 6-84. AOCS method Ba 6-84 is useful for grains, meals, flours, feeds and all fiber bearing material from which the fat can be extracted.leaving a workable residue. Crude protein content of corn meal is determined using AOCS method Ba 4e-93. The starch content of corn meal is determined using AOCS method Ba 4e-93. The starch content of corn meal is determined using the Standard Analytical Methods of the Member Companies of the Corn Refiners Association Incorporated, 2d Edition, Apr. 15, 1986, method A-20 (“Corn Refiner's method A-20”).

It is to be understood that the analytical methods provided herein are illustrative examples of useful methods for computing various quality parameters for the oils and meals described herein. Other suitable methods are known and may be used to compute the quality parameters disclosed and claimed herein.

The following examples are included to demonstrate specific embodiments of the invention. It should be appreciated by those of skill in the art that the techniques disclosed in the examples which follow represent techniques discovered by the inventors to function well in the practice of the invention, and thus can be considered to constitute exemplary modes for its practice. However, those of skill in the art should, in light of the present disclosure, appreciate that many changes can be made in the specific embodiments which are disclosed and still obtain a like or similar result without departing from the spirit and scope of the invention.

Example 1 provides a detailed description of a solvent extraction-based method for extraction of oil from high oil corn to produce an extracted corn meal. Table 2 provides a detailed component profile of two different extracted corn meals. The corn meal with lower oil content was subjected to a greater degree of extraction than was the corn meal with the higher oil content.

Example 2 provides a detailed component and nutrient profile for a hog finishing feed ration made with the extracted corn meal as prepared in Example 1. Table 3 compares the feed ration made with the extracted corn meal to a feed ration made with conventional corn. Due to the particular nutrient profile of the extracted corn meal, the finishing feed ration made therefrom requires no conventional corn and less soybean meal as well as different amounts of other components, in order to provide a suitable feed ration.

Example 3 provides a detailed component and nutrient profile for a poultry finishing feed ration made with the extracted corn meal as prepared in Example 1. Table 4 compares the feed ration made with the extracted corn meal to a feed ration made with conventional corn. Due to the particular nutrient profile of the extracted corn meal, the finishing feed ration made therefrom requires no conventional corn and less soybean meal in order to provide a suitable feed ration.

Example 4 describes a process for obtaining a corn oil having an elevated tocotrienol content. The corn oil was made according to Example 1. Oil derived from the high oil corn is compared to commercially available crude oil, which is oil obtained by conventional, methods from conventional corn. The extracted oil comprises elevated levels of tocotrienols, in particular alpha- and gamma-tocotrienols.

Example 5 provides a detailed component and nutrient profile for a blended feed ration made from soybean meal and the extracted corn meal alongside the nutrient requirements for poultry and swine diets as set forth by the guidelines of the National Research Council (NRC). Unlike corn grain, the extracted corn meal when combined with soybean meal, prepared herein provides higher protein and amino acid levels and flexible oil levels to help meet nutrient levels required by the NRC.

### EXAMPLE 1

### Processing High Oil Corn Using Cracking, Conditioning and Flaking Method

A 45 pound sample of high oil corn was cracked using a Roskamp 6.5 Series (9″ two sets) set at a roll gap of 0.27 inches. A sample was taken for analysis and the remaining sample split into 4 sub-samples. Each of the four sub-samples was then conditioned independently to different temperatures (120 F, 150 F, 180 F, 200 F). The samples were heated in Crown 18 inch De-solventiser/Toaster. After each sample reached its conditioning temperature the samples were passed through flaking rolls. The flaking rolls used were a Ross 10 inch set to a gap of 0.007 inches. A sample of the flakes was taken and about a 500 gram sample was extracted. The flaked sample was washed for four 20 minute periods with 1200 ml of hexane each period for a total of 4800 ml of solvent over 120 minutes. The solvent temperature was about 120 F. The miscella was collected and filtered through #4 qualitative circles 185 mm diameter. The marc was air dried at room temperature. The filtered miscella was then roto-evaporated to estimate the percent oil recovery. Samples of the oil and meal were taken and analyzed for fatty acid profile, starch, protein and fiber. During the extraction a sieve analysis was performed and flake thickness was measured.

Other equipment used for the analysis included a Mettler Toledo HR73 Halogen Moisture Analyzer, Ohaus Explore scale, Büchi R-1 14 Roto-Vap, Crown extractor screen 0.032 sieve and a easy-load master Flex Model 7529-30 pump.

The color of the crude oil was visually evaluated and determined to be a light yellow color compared to a crude oil isolated using conventional wet milling methods, which was a dark brown color.

The desolventized corn meal was characterized using AOCS methods Ba 3-38, Ba 2b-82, Ba 6-84, and Ba 4e-93, and Corn Refmer's Method A-20. When normalized to a 10% moisture content, the corn meal had about 3.2% fiber content, about 65% starch content, and about 14% protein content. Meal fat was determined to be about 1.07% using AOCS method 3-38. For comparison, corn gluten feed created using conventional wet milling methods and normalized to a 10% moisture content can be expected to contain an oil content of about 4%, a protein content of about 20%, and a fiber and other carbohydrate content of about 60%. Also for comparison, corn gluten meal created using conventional wet milling methods and normalized to a 10% moisture content can be expected to contain an oil content of about 3%, a protein content of about 60%, and a fiber and other carbohydrate content of about 22%.

The nutrient profiles of two types of meal (1.5% oil and 4.0% oil) produced according to this process are shown in Table 2.

TABLE 2
Nutrient content of two meal types (1.5% oil and 4.0% oil) produced by
extraction of oil from high oil corn grain having 12% oil and 9% protein.
Amounts are expressed on an “as is” or “as fed” moisture level.

| | | Amount |
|---|---|---|
| Component | Amount (%) | (%) |
| Moisture | 12 | 12 |
| Oil | 1.5 | 4 |
| Protein | 10.5 | 10.2 |
| Starch | 58.0 | 56.3 |
| Neutral Detergent Fiber | 11.3 | 11 |
| Acid Detergent Fiber | 2.8 | 2.8 |
| Ash | 1.4 | 1.3 |
| Lysine | 0.39 | 0.37 |
| Tryptophan | 0.105 | 0.102 |
| Methionine | 0.29 | 0.28 |
| Cystine | 0.25 | 0.24 |
| Total Sulfur Amino Acids | 0.54 | 0.52 |
| Valine | 0.53 | 0.51 |
| Isoleucine | 0.40 | 0.39 |
| Arginine | 0.53 | 0.51 |
| Threonine | 0.40 | 0.39 |
| Leucine | 1.20 | 1.17 |
| Histidine | 0.32 | 0.31 |
| Phenylalanine | 0.51 | 0.5 |
| Alanine | 0.82 | 0.79 |
| Serine | 0.54 | 0.52 |
| True metabolizable energy | 3023 | 3133 |
| (TMEn; kcal/kg) | | |
| Swine metabolizable energy | 3191 | 3301 |
| (ME; kcal/kg) | | |

### EXAMPLE 2

### Use of Meal Derived From Corn Processed Through Flaking and Extraction as a Component of Hog Finishing Feed Ration

This example details a comparison of two different feed rations: a first feed ration containing normal corn that has not been solvent extracted and a second feed ration containing extracted corn meal. The feed ration containing extracted corn meal is used when lean pork meat is a desired end product. A hog finishing feed ration comprising an extracted corn meal containing less than or about 1.5% oil is prepared by providing the following ingredients in the amounts indicated in Table 3. The feed ration is generally produced by blending, mixing, and pelletting the ingredients to produce a feed product; however, one or more of these steps can be omitted in the process of preparing the feed ration.

TABLE 3
Comparison of swine feed rations made using normal corn
(not high oil corn) and extracted corn meal obtained from high
oil corn comprising 12% oil, 9% protein, wherein the extracted
corn meal has about 1.5% or less of oil (fat). Nutrient levels
are shown. Amounts are expressed on an “as is” or
“as fed” moisture level.

Swine Finishing Feed

| | Normal Corn | Extracted Corn |
|---|---|---|
| Ingredients | (%) | Meal (%) |
| Corn | 79.98 | — |
| Extracted corn meal | — | 83.55 |
| (about 1.5% oil) | | |
| Soybean meal | 12.45 | 6.60 |
| Meat & bone meal | 6.59 | 7.22 |
| Feather meal | — | — |
| Fat | 0.10 | 1.50 |
| Salt | 0.40 | 0.70 |
| Lysine | 0.08 | 0.15 |
| Methionine | — | — |
| Premix | 0.15 | 0.15 |
| Nutrient | | |
| Crude protein, % | 15.44 | 15.78 |
| ME, kcal/kg | 3200 | 3200 |
| Crude fiber, % | 1.96 | 2.12 |
| Calcium, % | 0.85 | 0.85 |
| Phosphorus, % | 0.58 | 0.58 |
| Amino Acids, % | | |
| Arginine | 0.96 | 0.93 |
| Cyctine | 0.28 | 0.29 |
| Histidine | 0.40 | 0.42 |
| Isoleucine | 0.57 | 0.58 |
| Leucine | 1.39 | 1.49 |
| Lysine | 0.81 | 0.81 |
| Methionine | 0.26 | 0.34 |
| Phenylalanine | 0.70 | 0.72 |
| Threonine | 0.56 | 0.58 |
| Tryptophan | 0.14 | 0.14 |
| Tyrosine | 0.47 | 0.48 |
| Valine | 0.72 | 0.75 |

In Table 3, absolute values for ingredient percentages are given, however, in practice, the ingredients could be included using the inclusion rates shown in other tables herein.

Some advantages of the new feed ration are that a user of the meal would not need to grind corn, thus saving an energy intensive step, less soybean or other oilseed meal is required to meet desired protein levels, and the meal may have better digestibility than corn grain.

### EXAMPLE 3

### Use of Meal Derived From Corn Processed Through Flaking and Extraction as a Component of Poultry Finishing Feed Ration

This feed ration is used to fulfill the high energy requirements of growing birds such as broilers. A poultry broiler finishing feed ration comprising an extracted corn meal containing less than or about 4% oil (fat) is prepared by providing the following ingredients in the amounts indicated in Table 4. The feed ration is generally produced by blending, mixing, and pelletting the ingredients to produce a feed product; however, one or more of these steps can be omitted in the process of preparing the feed ration.

TABLE 4
Comparison of poultry feed rations made using normal corn
(not high oil corn) and extracted corn meal obtained from
high oil corn comprising 12% oil, 9% protein, wherein the
extracted corn meal has about 4% or less of oil (fat).
Nutrient levels are shown. Amounts are expressed on
an “as is” or “as fed” moisture level.

Growing Broiler

| | Normal Corn | Extracted Corn |
|---|---|---|
| Ingredients | (%) | Meal (%) |
| Normal corn | 66.85 | — |
| Extracted corn meal | — | 70.86 |
| (about 4% oil) | | |
| Soybean meal | 20.96 | 16.42 |
| Meat & bone meal | 5.00 | 5.00 |
| Feather meal | 2.00 | 2.00 |
| Fat | 3.29 | 3.76 |
| Salt | 0.37 | 0.37 |
| Added Lysine | 0.13 | 0.19 |
| Added Methionine | 0.15 | 0.09 |
| Premix | 0.10 | 0.10 |
| Nutrient | | |
| Crude protein, % | 19.48 | 19.52 |
| ME, kcal/kg | 3100 | 3100 |
| Crude fiber, % | 1.97 | 2.12 |
| Calcium, % | 0.94 | 0.94 |
| Phosphorus, % | 0.63 | 0.62 |
| Amino Acids, % | | |
| Arginine | 1.27 | 1.23 |
| Cyctine | 0.38 | 0.39 |
| Histidine | 0.47 | 0.48 |
| Isoleucine | 0.78 | 0.79 |
| Leucine | 1.68 | 1.74 |
| Lysine | 1.06 | 1.06 |
| Methionine | 0.44 | 0.44 |
| Phenylalanine | 0.92 | 0.92 |
| Threonine | 0.74 | 0.75 |
| Tryptophan | 0.19 | 0.20 |
| Tyrosine | 0.61 | 0.62 |
| Valine | 0.95 | 0.96 |

In Table 4, absolute values for ingredient percentages are given, however, in practice, the ingredients could be included using the inclusion rates shown in other tables herein.

### EXAMPLE 4

### Use of Oil Derived From Corn Processed Through Flaking and Extraction as a Component of Food Products, or as a Starting Material for Purification of Kernel Components

In this example, oil with approximately a 200% to 300% increase in tocotrienol content over conventionally-produced crude corn oil is described. Using the method of flaking and extraction of Example 1, corn oil was extracted from high oil corn grain having an oil content of about 12%. The corn oil was then analyzed for tocotrienol content. The table below includes data concerning the alpha- and gamma-tocotrienol content of conventional corn oils produced by conventional processing of conventional corn and the extracted corn oil prepared according to the method of Example 1. Conventional Crude oil refers to an unrefined corn oil sample. The sample is representative of corn oil of the type that is most commonly produced presently. As noted below, the tocotrienol content of extracted whole kernel oil (EWKO) samples from two different high oil corn samples that were extracted with solvent at temperatures ranging from 120 to 200 degrees Fahrenheit was found to be approximately two to three times higher than in the conventional crude oil sample. The tocotrienol content of the EWKO samples ranged from about 26 ppm to about 33 ppm of alpha-tocotrienol and from about 48 ppm to about 84 ppm of gamma-tocotrienol. Generally, increasing the extraction temperature results in an increase in the tocotrienol content of the extracted corn oil. The actual minimum and maximum values for tocotrienol content will depend upon the particular high oil corn used.

| | alpha | Gamma |
|---|---|---|
| Sample | tocotrienol(ppm) | tocotrienol(ppm) |
| Conventional Crude Oil | 11.88 | 29.94 |
| (Control) | | |
| EWKO 1 120-200 F. | 29.36-33.19 | 48.11-59.36 |
| EWKO 2 120 F. | 26.05-28.43 | 79.55-84.21 |

Accordingly, the process of Example 1 is used to make an extracted corn oil comprising elevated levels of tocotrienols.

### EXAMPLE 5

### Use of Meal Derived From Corn Processed Through Flaking and Extraction as a Component of a Blended Animal Feed Product Comprised of Corn Meal and an Oilseed Meal

This example illustrates a novel feed ingredient comprised of a blend of a corn meal produced by the flaking and oil extraction method and another plant-based meal such as an oilseed meal. This blended material could be in the form of simply a loose agregate mixture of both meal types, or a pelletted product. Because the method for producing the corn and oilseed meals would be similar, i.e., cracking, conditioning, flaking and solvent extraction, it is possible to produce both meals in proximity and blend them prior to shipment to a customer. An advantage of this approach is that varying protein and energy levels can be created in a single meal. Additional ingredients are optionally added either at the meal blending stage or at a later time. For example, an energy-intensive step in feed manufacturing involves grinding corn grain and blending it with other ingredients at a feed mill. The present blended meal generally requires less energy to produce a finished feed product than does a conventional blended meal.

Table 5 shows nutrient profiles of soybean meal (SBM), extracted corn meal (ECM), a blend of 20% SBM and 80% ECM (S20-C80), a blend of 10% SBM and 90% ECM (S10-C90), and nutrient requirements for poultry and swine diets. The poultry and swine nutrient requirements shown are in accordance with National Research Council (NRC) guidelines. The ECM was prepared according to Example 1.

| | | | | | | Swine |
|---|---|---|---|---|---|---|
| | | | | | | Requirement |
| | | | | Poultry | | NRC |
| | | | | Requirement | | For Finishing |
| | | | S20-C80 | NRC | S10-C90 | Hog |
| Parameter | SBM | ECM | Level | Level | Level | Level |
| Crude Protein (CP) | 47.5 | 10.2 | 17.66 | 18 | 13.93 | 13.2 |
| Swine ME, kcal/kg | 3380 | 3301 | 3316.8 | | 3308.90 | 3265 |
| Poultry ME, kcal/kg | 2440 | 3133 | 2994.4 | 3200 | 3063.70 | |
| Crude Fat, % | 3 | 4 | 3.8 | | 3.90 | |
| Neutral Detergent | 8.9 | 11.3 | 10.82 | | 11.06 | |
| Fiber, % | | | | | | |
| Acid Detergent Fiber, % | 5.4 | 2.8 | 3.32 | | 3.06 | |
| Arginine | 3.48 | 0.45 | 1.06 | 1.00 | 0.75 | 0.19 |
| Histidine | 1.28 | 0.27 | 0.47 | 0.27 | 0.37 | 0.19 |
| Isoleucine | 2.16 | 0.34 | 0.70 | 0.62 | 0.52 | 0.33 |
| Leucine | 3.66 | 1.03 | 1.56 | 0.93 | 1.29 | 0.54 |
| Lysine | 3.02 | 0.33 | 0.87 | 0.85 | 0.60 | 0.60 |
| Methionine | 0.67 | 0.25 | 0.33 | 0.32 | 0.29 | 0.16 |
| Cystine | 0.74 | 0.21 | 0.32 | 0.28 | 0.26 | 0.35 |
| Phenylalanine | 2.39 | 0.44 | 0.83 | 0.56 | 0.64 | 0.34 |
| Tyrosine | 1.82 | 0.29 | 0.60 | 0.48 | 0.44 | 0.55 |
| Threonine | 1.85 | 0.34 | 0.64 | 0.68 | 0.49 | 0.41 |
| Tryptophan | 0.65 | 0.09 | 0.20 | 0.16 | 0.15 | 0.11 |
| Valine | 2.27 | 0.45 | 0.81 | 0.70 | 0.63 | 0.40 |
| Total Essential Amino | 23.99 | 4.49 | 8.39 | 6.85 | 6.44 | 4.17 |
| Acids (EAA) | | | | | | |
| EAA/CP | 0.505 | 0.440 | 0.45 | 0.381 | 0.45 | 0.316 |

### EXAMPLE 6

### Processing High Oil Corn Using Flaking Method

Shelled kernels of individual ears of yellow dent corn were screened for a total oil content greater than about 7% oil using a Perten bulk near infrared (NIR) seed tester (TM) (model 9100-H.F) Perten Instruments, P.O. Box 7398, Reno, Nev. 89510. Kernels from the ears having at least a 7% oil content were screened further for individual kernels having an oil content of at least 13% oil in a Brimrose seedmeister (TM) single kernel NIR tester (Brimrose Corp., Baltimore, Md.). The kernels were stored at a moisture content of about 13.5%. At the time of processing, the moisture content of the seed was about 10%.

A bench scale flaking apparatus containing a two-inch stainless steel rod and plate was used to flake the whole corn grain. The whole corn grain sample was passed through the rollers four times to obtain a final flake thickness of about 0.01 inches. A miscella was extracted from the flaked corn grain using hot (60-65EC) n-hexane and a Kimble (TM) model 585050 Soxhlet extractor. The resulting miscella and corn meal were desolventized. The miscella was desolventized by heating the miscella to 70EC under a vacuum of 25 inches mercury. The corn meal was desolventized according to AOCS method Ba 2a-38.

The total recovered oil was determined to be 14.74% of the whole corn grain sample. The phosphorus content of the desolventized crude oil was determined to be 365 parts per million (ppm) using AOCS method Ca 12-55. The phospholipid concentration was determined to be 1.095% (0.0365%*30). The free fatty acid content was determined to be 0.2% using AOCS method Ca 5a-40. The neutral oil loss during processing was determined to be 1.3% (1.095%+0.2%). Using the same methods, crude oil extracted from normal, i.e., 3-4% total oil content, corn grain using conventional wet milling methods can be expected to have a phosphorous content from about 600 ppm to about 800 ppm, a free fatty acid concentration from about 0.5% to about 1.0 percent, and a neutral oil loss during processing ranging from about 3% to about 4%.

The color of the crude oil was visually evaluated and determined to be a light yellow color compared to a crude oil isolated using conventional wet milling methods, which was a dark brown color.

The desolventized corn meal was characterized using AOCS methods Ba 3-38, Ba 2b-82, Ba 6-84, and Ba 4e-93, and Corn Refiner's Method A-20. When normalized to a 10% moisture content, the corn meal had a 3.2% fiber content, a 65% starch content, and a 14% protein content. Meal fat was determined to be 1.07% using AOCS method 3-38. For comparison, corn gluten feed created using conventional wet milling methods and normalized to a 10% moisture content can be expected to contain an oil content of about 4%, a protein content of about 20%, and a fiber and other carbohydrate content of about 60%. Also for comparison, corn gluten meal created using conventional wet milling methods and normalized to a 10% moisture content can be expected to contain an oil content of about 3%, a protein content of about 60%, and a fiber and other carbohydrate content of about 22%.

To the extent not already indicated, it also will be understood by those of ordinary skill in the art that any one of the various specific embodiments herein described and illustrated may be further modified to incorporate features shown in other of the specific embodiments.

Unless otherwise specified, the weights or percentages indicated herein are on a dry weight basis. As used herein, percentages are expressed in w/w ratios. The artisan of ordinary skill will recognize that when amounts of ingredients are expressed herein as ranges in compositions, the ingredients are generally present such that not all ingredients are necessarily present at their respective maximum concentrations. Rather, any one or more ingredients may be present at their respective maximums and the amounts of the remaining ingredients would be adjusted such that the total of all ingredients in a given composition does not exceed 100% wt.

The foregoing detailed description has been provided for a better understanding of the invention only and no unnecessary limitation should be understood therefrom as some modification will be apparent to those skilled in the art without deviating from the spirit and scope of the appended claims. As such, other aspects, advantages, and modifications are within the scope of the following claims.

1-35. (canceled)

36. A method of using extracted corn meal in an animal feed ration comprising the steps of:
providing an extracted corn meal prepared by at least flaking high oil corn to form flaked corn and extracting the flaked corn to remove a portion of the corn oil therefrom; and
including the extracted corn meal in an animal feed ration.

37. The method of claim 36, wherein the high oil corn has been conditioned prior to flaking to form a conditioned high oil corn.

38. The method of claim 37, wherein the conditioned high oil corn has been cracked prior to conditioning.

39. The method of claim 36, wherein a major portion of the oil originally present in the high oil corn is removed by extraction.

40. The method of claim 36, wherein at least 10% weight of the oil originally present in the high oil corn is removed by extraction.

41. The method of claim 36, wherein the extracted corn meal comprises:
about 0.75-12.0 wt.% of fat;
about 5-45 wt.% of moisture;
about 7-20 wt.% of protein;
about 2-4 wt.% of crude fiber; and
about 40-80 wt.% of carbohydrate.

42. The method of claim 41, wherein the majority of the animal feed ration is the extracted corn meal.

43. The method of claim 41 further comprising the step of:
adding to the extracted corn meal at least one other component selected from the group consisting of grain, flavorant, preservative, oil, oilseed meal, meat & bone meal, protein, fiber, feather meal, fat, salt, amino acid, vitamin and mineral to form the animal feed ration.

44. The method of claim 43 further comprising the step of:
providing the animal feed ration as a finishing feed ration for at least one of swine, layer poultry and broiler poultry.

45-73. (canceled)

74. A method of using a corn meal in an animal feed or human food, wherein the corn meal is obtained after extraction of corn oil from whole kernels of high oil corn, the method comprising the step of including the corn meal in an animal feed or human food.

75. (canceled)
